(19) United States
(12) Patent Application Publication
(10) Pub. No.: US 2006/0293623 A1
(43) Pub. Date: Dec. 28, 2006

(54) Orthopedic restraint and method for shoulder remediation

(76) Inventor: Michael F. Carroll, Traverse City, MI (US)

Correspondence Address: Douglas S. Bishop;Bishop & Heintz, P.C., P.O. Box 707, Traverse City, MI, 49685-0707, US

(21) Appl. No.: 11/159,942

(22) Filed: Jun. 23, 2005

(51) Int. Cl. A61F 5/00

(52) U.S. Cl. 602/20; 602/23; 602/19

(57) ABSTRACT

An orthopedic treatment device and method to prevent impingement of a patient's supraspinatus tendon between the head of the humerus and the coracoacromial arch in the patient's shoulder, while the patient is sleeping, or otherwise in a supine or prone position. The invention inhibits abduction of the arm. This limitation of the arm away from the body prevents the stated impingement of the supraspinatus tendon. The device is designed for treatment of rotator cuff tendonitis. This impingement is also prevented by direct traction exerted on the arm in a downward direction by the invention. The device consists of a first sleeve removably fixed around the patient's thigh and a second sleeve removably fixed around the patient's wrist, both proximate the shoulder treated. The sleeves are connected by a resilient strap which exerts traction on the arm.

### FIELD OF THE INVENTION

This invention relates to medical treatment or rehabilitation devices, generally, and, more specifically, to orthopedic treatment devices for maintaining traction and preventing impingement in a patient's shoulder. While the present invention may have application to different medical conditions, it is particularly adapted to treatment of what is commonly known as rotator cuff tendonitis or impingement syndrome.

### BACKGROUND OF THE INVENTION

In the United States, shoulder pain is currently the third most common cause of musculoskeletal disorder following low back pain and cervical pain. Some estimates of the cumulative incidence of shoulder disorders in the general western population have varied from 7% to 25%. The annual incidence has been estimated at 10 cases per 1,000 population, peaking at 25 cases per 1,000 population in the age category of 42 to 46 years of age. In the segment of the population of age 70 years or older, 21% of persons in this category have been found to have shoulder symptoms, most of which have been attributed to rotator cuff disorder or rotator cuff tendonitis.

Although often considered a benign condition, previous studies on the long term outcome of rotator cuff tendonitis have demonstrated that a significant majority of patients are still symptomatic at 18 months, even after receiving what was considered sufficient conservative treatment. Many of these patients rate their symptoms as severe.

Musculoskeletal disorders are the primary disabling conditions of working adults. The prevalence of rotator cuff tendonitis has been found to be as high as 18% in certain groups of workers who performed heavy manual labor.

Within the shoulder structure, impingement upon the supraspinatus tendon between the head of the humerus and the coracoacromial arch is thought to be the primary cause of rotator cuff tendonitis. This process then leads to degeneration and further disease of the supraspinatus tendon and subsequently the complex rotator cuff. Thus, reduction of movement which causes impingement on the supraspinatus tendon must then be assumed to reduce its degeneration. Maximum impingement occurs when a person's arm is in abduction, or away from the body. Abduction in this manner frequently occurs in circumstances when the body is sleeping. Today's sleeping surfaces, with firm support, maintaining the body in a relatively flat position produces circumstances where a person's arms are frequently in an abducted position. Clearly, a device and method to limit arm abduction and/or shoulder impingement, particularly directed to sleeping positions, or when the body is otherwise in a supine or prone position, is of great value.

Impingement becomes less of a potential problem when the body is upright, because the inherent weight of a person's arm provides necessary and constant traction to keep the head of the humerus unimpinged. (Another name for “rotator cuff tendonitis” is “impingement syndrome”.) During the nighttime, a person's shoulder, in normal sleep or otherwise, when in a supine, or prone, position, loses the built-in traction protection provided by gravity.

The ability to immobilize a shoulder joint is known. U.S. Pat. No. 5,423,333, to Jenson et al, provides an example of an apparatus for immobilizing a human shoulder and for supporting the wrist of the arm associated with that shoulder; U.S. Pat. No. 5,569,172, to Padden et al, provides a device for supporting and immobilizing a patient's arm and shoulder by padding and strapping the same to the user's torso. Other examples of prior art may be found wherein pressure is exerted on the shoulder itself, to hold it in place, or to inhibit movement of a person's arm proximal the affected shoulder.

U.S. Pat. No. 4,610,244, to Hammond, provides a brace which limits motion of the shoulder. The device of the Hammond patent, however, appears to potentially cause an upward force upon the arm, as the fixation point on the user's forearm is matched to a torso strap affixed around the waist. While the '244 patent discloses a device which can prevent abduction of the wrist and arm, it is not directed to preventing impingement in the shoulder, when the patient is lying down or sleeping. It does not disclose the ability to prevent natural impingement in a lying or sleeping position, nor does it disclose the ability to provide a gravity replacing, tractional “pull” to prevent impingement.

Further, none of the prior art appears adapted to a device which would be comfortable to the user, to hold the entire arm in a position substantially similar to a suspended at rest arm position, as provided by gravity when the user is upright, to prevent abduction, and impingement, while being worn at night, while sleeping, or otherwise in a supine, or prone position.

The prior art fails to provide a device to be worn at night, or at other times when the body is lying down, or otherwise in a prone, or supine, position, which device may be comfortably attached to a patient's upper and lower proximal extremities in a manner to hold the arm in a position similar to that of the arm when at rest and suspended as a result of gravity, and which device would provide a dual function of preventing undue abduction of the patient's proximal arm and, at the same time, when movement of the proximal arm is attempted, providing a reactive tractional force away from the shoulder joint, in a situation where gravity is not available to provide natural traction, further limiting or preventing impingement upon the supraspinatus tendon.

The present invention addresses this long-felt need, among others, as further hereinafter described.

As noted, existing, singular medical devices and techniques fail to satisfy these requirements.

### SUMMARY OF THE INVENTION

The present invention has been designed to overcome the shortcomings in the prior art noted above.

The invention is directed to provision of a remedial orthopedic device, generally, for any condition in which the prevention of abduction of the proximal arm and/or prevention of impingement of the shoulder joint is deemed beneficial, and, more specifically, for said prevention of abduction, and impingement in treating rotator cuff tendonitis/impingement syndrome.

The invention provides an orthopedic remedial medical device which may be comfortably worn by a patient at all times when the patient is sleeping, or otherwise in a supine, or prone, position.

The invention limits abduction of the proximal arm to the shoulder being treated and, further, provides, through the elastic strap, an inherent tractional force. When the device is properly applied, with the strap under some degree of expansion and, accordingly, tension, the tractional force is immediately and constantly present. Further, any reaction, in the way of movement of the wrist or arm, away from the point of attachment to the proximate thigh, creates a further reactive tractional force. Even in a circumstance where the device had not been applied to have an immediate tension on the elastic, or where the arm is further moved toward the proximate thigh to relieve the tractional force, a reversal of that movement recreates the tractional force, as soon as tension is present in the elastic strap.

Among other benefits, the invention is a remedial device, which may be incorporated within a method, which provides for a device which may be easily removed by conscious effort of the patient, utilizing the non-proximal hand, if movement of the arm should become necessary for other purposes.

In summary of the present invention, in its base form, the invention consists of a first removable sleeve about the proximal wrist of the patient, a second removable sleeve about the proximal thigh of the patient, and a resilient, elastic strap, of limited length, joining the two sleeves. When the device is in place, the user's wrist and, accordingly, proximal arm, along its length, are held closely, in an non-abducted position, to the body, with the arm fully extended, in a manner approximating the natural traction provided by gravity when the arm is in its natural suspended position while the person is upright. The wrist is, optimally, held in static position no more than several inches from the user's thigh, with the elastic strap under some degree of expansion/tension, thus providing a constant tractional force. If further movement is attempted, the further elasticity of the strap provides an additional contravening force pulling the wrist, and, consequently, the arm, toward its original position and increasing the tractional pull.

The sleeve elements may be permanently tubular in construction, with the patient's hand and wrist insertable through one end of one sleeve, and the foot, calf and thigh insertable through one end of the other, or, in practice, each sleeve, optimally, may be constructed of one rectangular piece of fabric or other like material. The rectangular material, at its greatest length, is then wrapped around the respective extremity (the wrist or thigh) and held in place by fasteners, which may be by buttons, ties, Velcro® type hook and loop fasteners, or other like devices.

The position of the device may be adjusted, and tension on the strap correspondingly adjusted, by relative positioning of one sleeve element on the patient's wrist or forearm, and the relative concurrent positioning of the other sleeve element at different positions on the patient's thigh. Further adjustment may be provided by having the elastic, resilient strap being adjustable, by buckle, buttons, ties, Velcro® type hook and loop fasteners or other like devices.

The above and additional features of the invention may be considered and will become apparent in conjunction with the drawings, in particular, and the detailed description which follow.

### BRIEF DESCRIPTION OF THE DRAWINGS

The following detailed description is understood by reference to the following drawings:

FIG. 1 is a perspective view of a portion of a patient's torso, and proximal arm and thigh showing the preferred embodiment of the invention in place.

FIG. 2 is a top view of the preferred embodiment of the invention, showing the sheet sleeve elements in open and in closed position.

FIG. 3 is a perspective view of the invention with the sleeve elements in formed and fastened position, and showing an adjustable strap element.

### DETAILED DESCRIPTION OF THE PREFERRED EMBODIMENT

Throughout the following detailed description, like numerals are used to describe the same element of the present invention shown in multiple figures thereof.

The invention, Orthopedic Restraint and Method for Shoulder Remediation, is an orthopedic appliance which limits abduction of the arm proximate the shoulder being treated and prevents impingement of a patient's (user) supraspinatus tendon between the head of the humerus and the coracoacromial arch in the treated shoulder.

Broadly considered, the device 10 is comprised of a retractably expandable strap 20, a first sleeve member 30, and a second sleeve member 40.

Strap 20 is made of elastic or other resilient construction, so that it is biased to return to its retracted state and, in its retracted state, it has a defined length 21. Strap 20 has a first has a first end 22 and a second end 23.

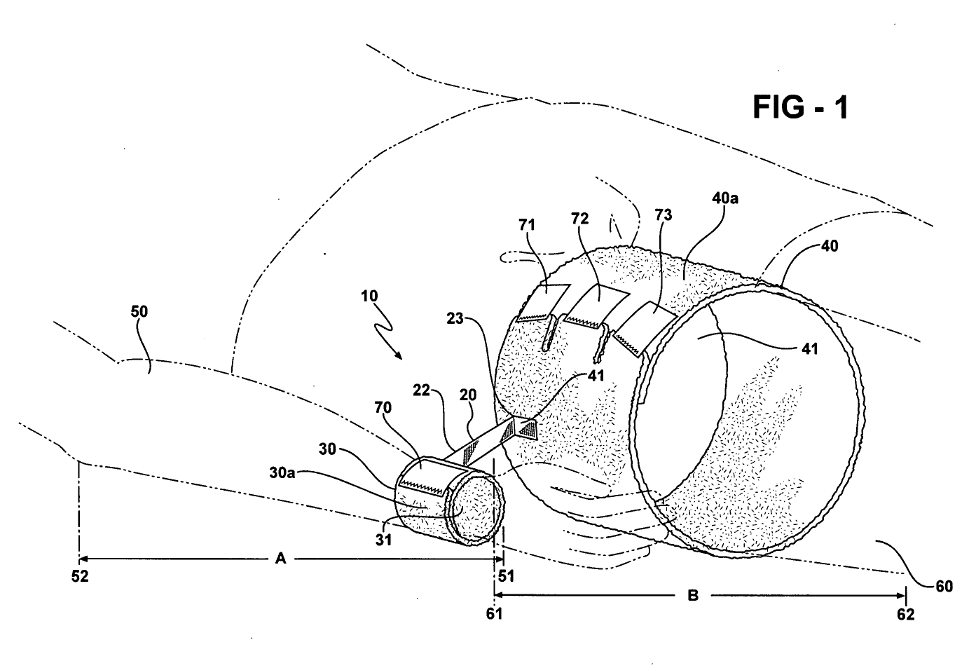

End 22 is connected to sleeve 30 by a connector 31 and to sleeve 40 by a connector 41. These connectors 22 and 23 may be sown thread, as shown in FIGS. 1, 2, and 3, or, alternatively, an adhesive bonding agent, buttons, rivets, buckles or like devices.

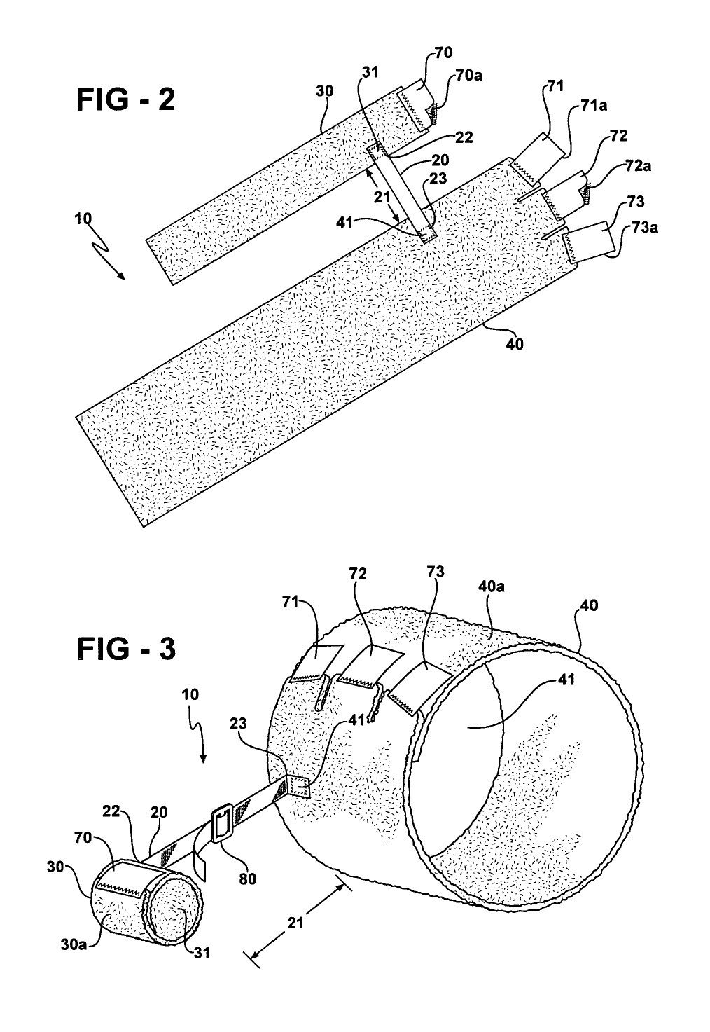

The sleeve members 3 and 4 are each constructed of pliable sheet material, which may be of rectangular shape as shown in FIG. 2 for both sleeves. However any shape which may be wrapped about the respective extremity will suffice. Each sleeve 3 and 4 has an open, or unwrapped, position as shown in FIG. 2, and a closed, wrapped, position as demonstrated in FIGS. 1 and 3.

The first sleeve 30 is wrapped around the wrist portion of the user's arm 50, which is connected to the shoulder being treated. The second sleeve 40 is wrapped around the user's thigh proximate (closest) to the arm 50. This alignment of the device as properly attached to the user's arm 50 and thigh 60 is demonstrated in FIG. 1.

For optimal effect when in use, strap 20, when retracted as shown in FIG. 2, is of a length 21 no greater than the distance between the user's wrist 50 and adjacent thigh 60, proximate the shoulder being treated, when the user is in a standing position and the arm 50 is suspended by gravity in its natural position. In practice, the length 21 will usually be between 0.0 and 8.0 inches.

The device 10 is preferably attached so that sleeve 30 is positioned on the user's wrist 50 within a range A between the hand 51 and elbow 52, and sleeve 40 is positioned on the user's thigh 60 within a range B between the user's hip 61 and knee 62, so that the strap 20 is taut as shown in FIG. 1 and under some degree of tension from the retractable expansion properties of the elastic or other like material, or strap 20 construction.

Each of sleeve 30 and sleeve 40 is held in its wrapped position by one or more fasteners 70, 72, 73, 74, as shown in FIGS. 1-3. Sleeve 30 is held in place by fastener 70 while sleeve 40 is held in position by fasteners 71, 72 and 73. As shown, fasteners 70, 71, 72 and 73 are Velcro® type hook and loop fasteners, with fasteners 70, 71, 72 and 73 containing the “hook” material 70a, 71a, 72a, 73a and the outer surface 30a of sleeve 30 and outer surface 40a of sleeve 40 being constructed of corresponding “loop” material. In alternative construction options, the sleeves 30 and 40 may be held in closed, wrapped position by other fasteners, including, but not limited to, buttons, buckles, external straps, or separate “hook and loop” corresponding patches connected at different intervals on the exterior surfaces of 30a and 40a of the sleeve members 30 and 40. Alternatively, the sleeve members 30 and 40 may be of unitary, tubular construction and attached by inserting the hand 51 and arm 50 of the user through the opening 31 of sleeve 30 and by inserting the full leg and thigh 60 of user through opening 41 of sleeve 40.

Sleeve 30 and sleeve 40 are constructed of fabric or like material. Such fabric is optimally of padded cotton composition to allow snug attachment while remaining comfortable to the user without limiting the invention. The device may also be used with different material with expandable properties or with expandable fasteners in place of fasteners 70, 71, 72 and 73.

Although the elastic nature of the strap 20 will normally make a standard strap 20 of static or retracted length 21 adaptable to most users, a buckle 80, or other adjuster, may be used to adjust the length 21 of strap 20, if desired.

As demonstrated, use of the above device 10, as described above, provides a novel method for treating disorders of the shoulder where it is desirable to limit abduction of the proximate arm 50, generally, and, in particular, where a significant inhibition of such abduction, to 10 degrees or less, is desirable.

In such method, the device 10, as described in detail above, is provided.

The device 10 is placed as shown in FIG. 2, with sleeves 30 and 40 in their open, unwrapped, position. Sleeve 30 is wrapped around the wrist portion of the user's arm 30 and proximate the treated shoulder and sleeve 40 is wrapped around the proximate thigh 60 of the user. Sleeve 30 is positioned within range A on the wrist portion of the user's arm 50 relative to sleeve 40 which is positioned within range B on the user's thigh 60 so that strap 20 is not fully retracted and, thus, is under some amount of tension, as shown in FIG. 1. When placed in their closed, wrapped position about the wrist portion of the arm 50 and the thigh 60, respectively, fastener 70 is used to fix sleeve 30 in its closed position and fasteners 71, 72 and 73 are used to fix sleeve 40 in its closed position.

It is the claims appended hereto, and all reasonable equivalents thereof, which define the true scope of the invention, and the invention is not limited to the depicted embodiments and exemplification.

What is claimed is:

1. An orthopedic device to limit abduction of the proximate arm and to prevent impingement in the user's shoulder, comprising:
a retractably expandable strap of defined length having a first end and a second end;
a first connector for joining said first end of said strap to the user's wrist proximate said shoulder; and
a second connector for joining said second end of said strap to the user's thigh proximate said shoulder;

2. The invention of claim 1, wherein the length of the retractably expandable strap is within a range of 0.0 to 8.0 inches in its retracted position.

3. The invention of claim 1, wherein the length of the retractably expandable strap, when restracted, is no greater than the distance between the user's wrist and thigh when the user's proximate arm is in its natural suspended position.

4. The invention of claim 1, wherein the connector for connecting the first end of said strap to the user's wrist is a first sleeve which removably wraps said wrist affixed to said first end and the connector for connecting the second of said strap to the user's thigh is a second sleeve which removably wrap said thigh, affixed to said second end.

5. The invention of claim 4, wherein said first sleeve and second sleeve are pliant sheet members which additionally comprise a fastener to hold them in their respective removably wrapped positions.

6. The invention of claim 5, wherein said first sleeve and second sleeve are constructed of fabric, and said fastener is one or more Velcro® type, hook and loop fasteners.

7. An orthopedic device to limit abduction of the proximate arm and to prevent impingement of the user's shoulder comprising:
a retractably expandable strap member of defined length, having a first end and a second end;
a first sleeve member which removably wraps the user's proximate wrist, affixed to the first end of said strap member; and
a second sleeve member which removably wraps the user's proximate thigh, affixed to the second end of said strap member.

8. The invention of claim 7, wherein said first sleeve member and second sleeve member are pliant sheets, each having an open, unwrapped position and a closed, wrapped position around the user's respective wrist and thigh.

9. The invention of claim 8, wherein said first sleeve member and second sleeve member are held in their respective closed positions by a fastener.

10. The invention of claim 9, wherein said first sleeve member and second sleeve member are constructed of fabric and said fastener is one or more Velcro® type, hook and loop fasteners.

11. The invention of claim 7, wherein said expandable strap may be held in expanded position by relative positioning of the first sleeve member and second sleeve member on the user's respective wrist and thigh.

12. The invention of claim 7, wherein the length of the retractably expandable strap, when retracted, is no greater than the distance between the user's wrist and proximate thigh when the user's proximate arm is in natural suspended position.

13. A method for preventing proximal arm abduction and impingement of a person's shoulder, when the person is sleeping or otherwise in a prone or supine position, comprising the following steps:
A. Providing an orthopedic remedial device comprising:
a retractably expandable strap member of defined length, having a first end and a second end;
a first sleeve member which removably wraps the user's proximate wrist, affixed to the first end of said strap member; and
a second sleeve member which removably wraps the user's proximate thigh, affixed to the second end of said strap member.
B. Affixing the first sleeve member around the person's proximate wrist;
C. Affixing the second member around the person's proximate thigh.

14. The method of claim 13, wherein the fist sleeve member and second sleeve member are positioned relative to each other in steps B and C to provide that the retractably expandable strap is under tension.

15. The method of claim 13, wherein said first and second sleeve member provided in Step A are pliant sheets, each having an open unwrapped position and a closed wrapped position around the user's respective waist and thigh.

16. The method of claim 13, wherein said first sleeve member and second sleeve member provided in Step A are held in their respective closed positions by a fastener.

17. The method of claim 16, wherein said first sleeve member and second sleeve member provided in Step A are constructed of fabric and said fastener is one or more Velcro® type hook and loop fasteners.

18. The method of claim 13, wherein the length of the retractably expandable strap provided in Step A is within a range of 0.0 to 8.0 inches in its retractable position.

19. The method of claim 13, wherein the length of the retractably expandable strap provided in Step A, when retracted, is no greater than the distance between the user's wrist and thigh when the user's proximate arm is in its natural suspended position.
